(19) United States
(12) Patent Application Publication
(10) Pub. No.: US 2018/0188202 A1
(43) Pub. Date: Jul. 5, 2018

(54) NOx SENSOR WITH CATALYTIC FILTER AND POLARISATION

(71) Applicant: ARMINES-ASSOCIATION POUR LA RECHERCHE ET LE DEVELOPPMENT DES METHODES ET PROCESSUS INDUSTRIELS, Paris Cedex 06 (FR)

(72) Inventors: Jean-Paul VIRICELLE, SURY LE COMTAL (FR); Philippe VERNOUX, ROCHETAILLEE SUR SAONE (FR); Christophe PIJOLAT, SAINT-ETIENNE (FR); Philippe BREUIL, SORBIERS (FR)

(21) Appl. No.: 15/737,681

(22) PCT Filed: Jun. 17, 2016
(86) PCT No.: PCT/EP2016/064046

§ 371 Date: Dec. 18, 2017

(30) Foreign Application Priority Data
Jun. 18, 2015 (FR) 1555600

(51) Int. Cl. G01N 27/407; G01N 33/00

(52) U.S. Cl. CPC G01N 27/4074; G01N 33/0037; G01N 27/4077

(57) ABSTRACT

A NOx gas sensor for placing in a gas stream of a heat engine exhaust device. The sensor includes a sensing element having a solid electrolyte with a first surface on which there extend a work electrode, a reference electrode, and a counter-electrode. The work electrode being made of a material having electrocatalytic and catalytic activity that are less than the electrocatalytic and catalytic activity of the materials of the reference electrode and of the counter-electrode, and the counter-electrode and the sensor include a catalytic filter surrounding the sensing element and bias means for negatively polarizing the work electrode. A detector device comprising a plurality of sensors of this type. A corresponding catalytic exhaust device.

The present invention relates to detecting nitrogen oxides (NOx) in a gas stream, and more particularly in the exhaust gas of a heat engine such as an internal combustion engine.

### STATE OF THE ART

It is known that the presence of excess nitrogen oxide in the atmosphere is a source of pollution and of health problems. Unfortunately, the operation of the heat engines, as used in particular for driving motor vehicles, generates nitrogen oxides that are released into the atmosphere. Catalytic exhaust devices are therefore generally associated with heat engines in order to trap the nitrogen oxides and minimize discharge into the atmosphere; provision is also made to adjust the operating parameters of the engine so as to maximize the efficiency of the catalytic exhaust device.

There has been a large amount of research on electrochemical sensors of NOx gases that are suitable for monitoring the emission of such pollutants. Such sensors may be arranged in a gas stream in catalytic exhaust devices of heat engines.

Such a sensor generally comprises a sensing element comprising a solid electrolyte having a first surface on which there extend a work electrode, a reference electrode, and a counter-electrode. The electrodes are electrically connected to a processor unit arranged to calculate gas concentration.

The choice of materials for the electrolyte and for the electrodes is determined as a function of the compounds to which the sensor is to be sensitive and as a function of its expected selectivity, i.e. the ability of the sensor not to be influenced by the presence of other compounds in the gas under analysis. For nitrogen oxides, a sensor has thus been envisaged that comprises a solid electrolyte made of yttria-doped zirconia and electrodes made of platinum.

Nevertheless, in exhaust devices, the sensors are generally subjected to an environment that is aggressive because of the chemical composition of exhaust gas and because of its high temperature. This complicates determining which materials to use for the electrodes and affects the selectivity and the reliability of the sensors.

### OBJECT OF THE INVENTION

An object of the invention is to propose a sensor of nitrogen oxides that is reliable and robust.

### BRIEF SUMMARY OF THE INVENTION

To this end, the invention provides a NOx gas sensor for placing in a gas stream of a heat engine exhaust device. The sensor comprises a sensing element having a solid electrolyte with a first surface on which there extend a work electrode, a reference electrode, and a counter-electrode. The work electrode is made of a material having electrocatalytic and catalytic activity that are less than the electrocatalytic and catalytic activity of the material(s) of the reference electrode and of the counter-electrode, and the sensor comprises:
a catalytic filter surrounding the sensing element; and
bias means for negatively polarizing the work electrode so as to make it selective for detecting nitrogen dioxide.

The catalytic filter firstly converts the carbon-containing reducing gases (hydrocarbons, carbon monoxide or CO) into carbon dioxide (CO₂) and water (H₂O), which do not lead to any response from the sensor, and secondly it transforms the NO, NO₂, and NH₃ compounds into a mixture of NO and NO₂ of composition that depends on thermodynamic equilibrium, and thus on temperature, and also on the partial pressure of oxygen. Thus, at constant temperature and oxygen pressure, and regardless of the initial NO and NO₂ composition, the sensor gives the same response. Thus, in the absence of bias, the sensor makes it possible to provide a response that depends essentially on the concentration of nitrogen oxides in the gas under analysis. Negatively polarizing the work electrode gives rise to the following electrochemical reduction reactions:

½O₂+2e⁻→O²⁻

NO₂+2e⁻→NO+O²⁻

The bias also opposes electrochemical oxidation reactions. The sensor no longer responds to reducing gases, including NO and NH₃, and is therefore selective for nitrogen dioxide. The sensor thus makes it possible to determine the concentration of nitrogen monoxide and the concentration of nitrogen dioxide.

Other characteristics and advantages of the invention appear on reading the following description of particular, non-limiting embodiments of the invention.

### BRIEF DESCRIPTION OF THE FIGURES

Reference is made to the accompanying drawing, in which:

FIG. 1 is a diagrammatic side view of an exhaust device provided with a sensor of the invention;

FIG. 2 is a diagrammatic elevation view of such a sensor; and

FIG. 3 is a diagrammatic view of a detector device including two sensors of the invention.

### DETAILED DESCRIPTION OF THE INVENTION

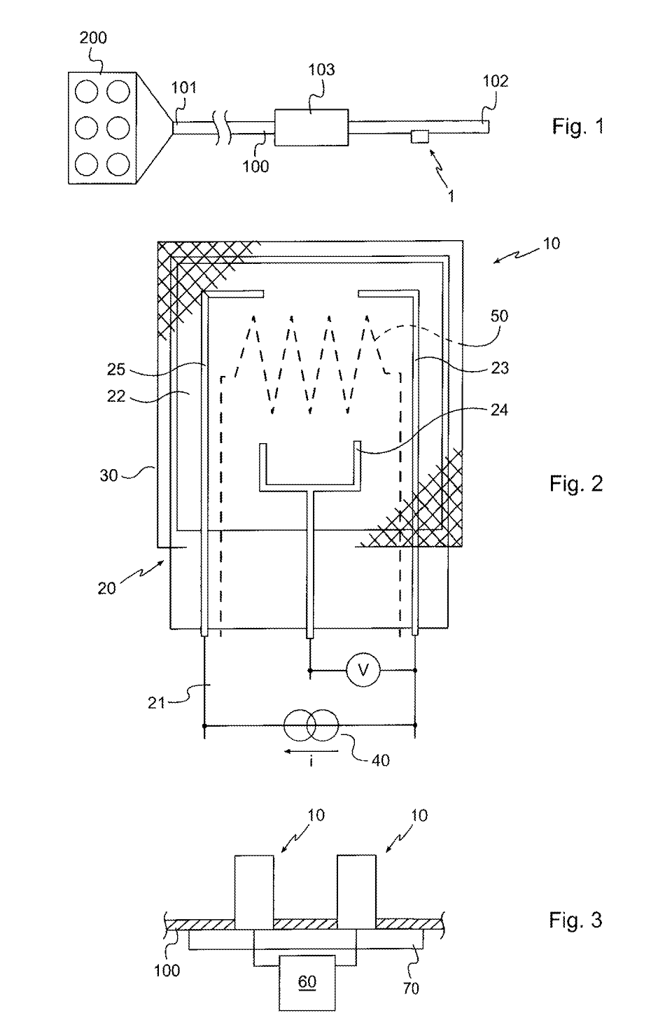

The invention is described herein in application to measuring nitrogen oxides in an exhaust device as shown in FIG. 1. The exhaust device comprises an exhaust system 100 having an inlet 101 connected to the exhaust manifold of an internal combustion engine 200 and an outlet 102 leading to the open air. The exhaust system 100 includes a known catalytic converter 103.

The nitrogen oxide detector device, given overall reference 1, is mounted in the exhaust system 100 downstream from the catalytic converter 103.

With reference to FIG. 2, the detector device 1 comprises a sensor given overall reference 10 in FIG. 2.

The sensor 10 comprises a sensing element 20 surrounded by a catalytic filter 30.

The sensing element 20 comprises a support 21 made of alpha alumina having a layer of yttria-doped zirconia deposited thereon so as to form a solid electrolyte 22. The solid electrolyte 22 may be made of some other material, and for example it may be made in any of the following materials: gadolinium-doped ceria (GDC), samarium-doped ceria (SDC), . . . .

The solid electrolyte 22 has a face on which there extend a work electrode 23, a reference electrode 24, and a counter-electrode 25. The work electrode 23 is made of a material having catalytic electrolytic activity that is less than the electrocatalytic and catalytic activity of the material of the reference electrode 24 and less than the electrocatalytic and catalytic activity of the material of the counter-electrode 25. The work electrode 23 includes at least an outer layer made of any one of the following materials: gold, ZnO, LaCrO₃, SnO₂, TiO₂, . . . . In this example, the work electrode 23 is made of gold. The reference electrode 24 and the counter-electrode 25 include at least an outer layer made of at least one of the following materials: platinum, nickel, rhodium, palladium, ruthenium, . . . . The reference electrode 24 and the counter-electrode 25 in this example are made entirely out of platinum.

The catalytic filter 30 has a porous substrate made of alpha alumina covered in at least one outer layer made of at least one of the following materials: platinum, nickel, rhodium, palladium, ruthenium, . . . . The outer layer in this example is made of platinum.

The sensor 10 has bias means 40 for negatively polarizing the work electrode 23. The bias means 40 are connected to the work electrode 23 and to the counter-electrode 25 in order to apply negative electrical bias to the work electrode 23.

A heater resistance 50 extends on the support 21 and is connected to a control unit 60 arranged to control the temperature of the sensing element 10. The heater resistance 50 extends over a face of the support 21 that is opposite from the electrodes 23, 24, and 25.

The detector device 1 is mounted on the exhaust system 100 in such a manner that the sensing element 20 surrounded by the catalytic filter 30 extends inside the exhaust system 100 in such a manner as to be in contact with the gas flowing through the exhaust system 100.

In operation, the catalytic filter 30 brings the set of NO and NO₂ compounds to thermodynamic equilibrium while conserving the total concentration of nitrogen oxide [NOx]ₜₒₜₐₗ. Once the bias has been applied, the concentration of NO₂ is measured, i.e. [NO₂]_{eq}, that results from this equilibrium:

$$NO + {}_{}^{1 / 2}{O}{}_{2}^{} = {NO}_{2}$$

$${K}_{T} = {{[ NO ]}_{{eq}^{*}} [ {O}_{2} ]}^{1 / 2} / {[ {NO}_{2} ]}_{eq}$$

$$\begin{matrix}{[ NO x ]}_{total} = [ NO ] + [ {NO}_{2} ] \\ = {[ NO ]}_{eq .} + {[ {NO}_{2} ]}_{eq} \\ = {( 1 + {{K}_{T}^{*} [ {O}_{2} ]}^{1 / 2} ) [ {NO}_{2} ]}_{eq}\end{matrix}$$

Thus the bias sensor with a filter gives the measurement [NO₂]_{eq} directly, so it is possible to calculate the concentration [NOx]ₜₒₜₐₗ by having knowledge of the thermodynamic constant K_{T} (thermodynamic table), and of the oxygen concentration [O₂]. As a result, negative bias is selective since it makes it possible to detect the concentration of nitrogen dioxide present among the nitrogen oxides.

If the oxygen concentration is not known by other means, it may be measured in the embodiment shown in FIG. 3: the detector device 1 comprises a support 70 having mounted thereon two sensors 10 that are identical to the above description (with filter and bias means) except in that they share a common control unit 60. The two sensors 10 are spaced apart from each other sufficiently for them to be subjected to a predetermined temperature difference (one sensor at a temperature T1 and the other sensor at a temperature T2). In this example, the temperature difference between T1 and T2 is about 20° C. to 30° C., and is preferably 20° C. Under such circumstances, the situation is as shown in the following formulas. Given that NO+½O₂=NO₂, the following apply:

at T1, K₁=[NO]_{1eq}*[O₂]₁^{1/2}/[NO₂]_{1eq}; and

at T2, K₂=[NO]_{2eq}*[O₂]₂^{1/2}/[NO₂]_{2eq}.

Since the nitrogen oxide concentration [NOx]ₜₒₜₐₗ is conserved from one sensor to the other, the following applies:

[NO]_{1eq}+[NO₂]_{1eq}=[NO]_{2eq}+[NO₂]_{2eq}

It is assumed that the dioxygen concentration is substantially the same from one sensor to the other, i.e. [O₂]₁≈[O₂]₂. This assumption is realistic given the orders of magnitudes of the concentrations: a few parts per million (ppm) for NO and NO₂ (maximum 1000 ppm), and a few percent for O₂ (where 1%=10,000 ppm).

It then follows that: [NO₂]_{eq}=S₁ and [NO₂]_{eq}=S₂, where S₁ and S₂ are the direct measurements from the sensors. This leads to a system of two equations in two unknowns that can easily be solved in order to obtain [NO]_{1eq} and [NO]_{2eq}:

S₁*K₁/S₂*K₂=[NO]_{1eq}/[NO]_{2eq}

[NO]_{1eq}*[1−(S₂*K₂)/(S₁*K₁)]=S₂−S₁

It is then easy to obtain the dioxygen [O₂] concentration and then the total concentration of nitrogen oxides [NOx]ₜₒₜₐₗ.

It should be observed that if the compound NH₃ is present in non-negligible quantity relative to NO and NO₂, and given that it is not destroyed by the filter but is retransformed into NO/NO₂, it will be incorporated in the [NOx]ₜₒₜₐₗ concentration.

Naturally, the invention is not limited to the embodiments described but covers any variant coming within the ambit of the invention as defined by the claims.

In particular, the detector device of the invention may be mounted in some other way in the exhaust system, and it is also usable in other applications.

In a variant of the second embodiment, if the oxygen concentration is known by other means (possible calculation of [NOx]ₜₒₜₐₗ) and if it is also desired to know [NO] and [NO₂] accurately on emission, it is possible to use a second sensor that does not have a catalytic filter but that has a negatively biased work electrode. The second sensor will then give the measurements of [NO₂] directly. It can then be deduced that [NO]=[NOx]ₜₒₜₐₗ−[NO₂].

1. A NOx gas sensor for placing in a gas stream of a heat engine exhaust device, the sensor comprising a sensing element having a solid electrolyte with a first surface on which there extend a work electrode, a reference electrode, and a counter-electrode, wherein the work electrode is made of a material having electrocatalytic and catalytic activity that are less than the electrocatalytic and catalytic activity of the material(s) of the reference electrode and of the counter-electrode, and in that the sensor comprises:
a catalytic filter surrounding the sensing element; and
bias means for negatively polarizing the work electrode so as to make it selective for detecting nitrogen dioxide.

2. The sensor according to claim 1, wherein the electrolyte is made of any of the following materials: yttria-doped zirconia, gadolinium-doped ceria (GDC), samarium-doped ceria (SDC).

3. The sensor according to claim 2, wherein the electrolyte is arranged on a support made of alpha alumina.

4. The sensor according to claim 1, wherein the work electrode includes at least an outer layer made of at least one of the following materials: gold, ZnO, LaCrO₃, SnO₂, TiO₂.

5. The sensor according to claim 1, wherein the reference electrode and the counter-electrode include at least an outer layer made of at least one of the following materials: platinum, nickel, rhodium, palladium, ruthenium.

6. The sensor according to claim 1, wherein the catalytic filter includes at least an outer layer made of at least one of the following materials: platinum, nickel, rhodium, palladium, ruthenium.

7. The sensor according to claim 6, wherein the outer layer covers a porous alumina substrate.

8. The sensor according to claim 1, including a heater resistance extending over a support to which the electrolyte is secured in order to control the temperature of the sensing element.

9. The sensor according to claim 8, wherein the heating resistance extends over a second face of the support opposite from the electrodes.

10. A detector device comprising a support having mounted thereon two sensors in accordance with claim 1, the two sensors being provided with means for controlling their temperatures and being far enough apart from each other for it to be possible for them to be subjected to a predetermined temperature difference.

11. The device according to claim 10, wherein the temperature difference lies approximately in the range 20° C. to 30° C.

12. A catalytic exhaust device including at least one sensor in accordance with claim 1.
